(19) United States
(12) Patent Application Publication
(10) Pub. No.: US 2024/0066290 A1
(43) Pub. Date: Feb. 29, 2024

(54) MEDICAL IMPLANT ELECTRODES WITH CONTROLLED POROSITY

(71) Applicant: Cochlear Limited, Macquarie University, NSW (AU)

(72) Inventors: Nicholas Charles Pawsey, North Ryde, NSW (AU); Jared Bergman, Sydney (AU)

(21) Appl. No.: 18/263,674

(22) PCT Filed: Feb. 2, 2022
(86) PCT No.: PCT/IB22/50922

§ 371 Date: Jul. 31, 2023

Related U.S. Application Data
(60) Provisional application No. 63/147,038, filed on Feb. 8, 2021.

(51) Int. Cl. A61N 1/05

(52) U.S. Cl. CPC A61N 1/0541

(57) ABSTRACT

An apparatus includes an electrode configured to be implanted on or within a recipient's body. The electrode includes a first portion having a surface configured to be in electrical communication with the recipients body and a second portion integral with the first portion and in mechanical and electrical communication with the first portion. The electrode further includes a plurality of pores extending from the surface of the first portion to the second portion such that the first portion has a substantially non-uniform porosity along a direction from the surface to the second portion.

### BACKGROUND

### Field

The present application relates generally to medical device electrodes configured to be implanted on or within a recipient's body, and more specifically to porous implantable electrodes.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises an electrode configured to be implanted on or within a recipient's body. The electrode comprises a first portion having a surface configured to be in electrical communication with the recipient's body. The electrode further comprises a second portion integral with the first portion and in mechanical and electrical communication with the first portion. The electrode further comprises a plurality of pores extending from the surface of the first portion to the second portion such that the first portion has a substantially non-uniform porosity along a direction from the surface to the second portion.

In another aspect disclosed herein, a method comprises forming a first portion of an electrode, the first portion comprising an electrically conductive material and having a first mass density. The method further comprises forming a second portion of the electrode, the second portion comprising the electrically conductive material, being integral with the first portion, and having a second mass density greater than the first mass density.

In another aspect disclosed herein, an apparatus comprises a porous electrode configured to be implanted on or within a recipient. The porous electrode has a surface region with a ratio of electrochemical surface area (ESA) to geometric surface area (GSA) greater than one. The surface region is configured to undergo dissolution over time while the porous electrode is implanted on or within the recipient with the ratio being substantially unchanged by the dissolution.

In another aspect disclosed herein, an electrode comprises a porous surface configured to be in electrical communication with a surrounding environment. The electrode further comprises a porous first metal portion at least partially bounded by the porous surface. The electrode further comprises a substantially non-porous second metal portion integral with the porous first metal portion and in mechanical and electrical communication with the porous first metal portion. The porous first metal portion has a porosity along a distance in a direction substantially perpendicular to the surface and extending from the porous surface to the substantially non-porous second portion. The porosity varies by more than 10% as a function of position along the distance, monotonically decreases along the distance, and has a non-zero and substantially continuous gradient as a function of position along the distance.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 is cross-sectional view of the cochlea illustrating the stimulating assembly partially implanted therein in accordance with certain implementations described herein;

FIG. 3 schematically illustrates a simplified side view of an example internal component comprising at least one stimulation electrode in accordance with certain implementations described herein;

FIGS. 4A-4C schematically illustrate cross-sectional views of various example apparatus in accordance with certain implementations described herein; and

FIGS. 5A-5C are flow diagrams of various example methods in accordance with certain implementations described herein.

### DETAILED DESCRIPTION

Medical device electrodes (e.g., cochlear implant electrodes) are expected to deliver stimulation over the lifetime of the recipient (e.g., 70 years or more). Even with charge balanced biphasic waveforms and use of inert materials (e.g., platinum), the electrodes can experience dissolution over these time periods, resulting in degradation or loss of function. Such dissolution can become more problematic for arrays in which smaller electrodes are used (e.g., to increase the number of electrodes of the array so as to increase the spectral resolution of the delivered signal). Furthermore, the risk of premature electrode dissolution can be exacerbated by the use of multipolar stimulation signals which entail higher charge levels and densities than do monopolar stimulation signals.

The electrodes of certain implementations described herein having a continuous and substantially non-uniform porosity (e.g., with at least some of the pores being closed, open, and/or interconnected with one another; the porosity having a gradient or being graded) along a direction extending from the electrode surface to deeper within the electrode. The electrode can be fabricated with a predetermined scale and/or nature of the porosity such that the substantially non-uniform porosity is tailored (e.g., optimized) for different purposes in different portions of the electrode. For example, the substantially non-uniform porosity can be decreasing (e.g., monotonically) along the direction and can increase the ration of electrochemical surface area (ESA) to geometric surface area (GSA) of an electrode surface configured to be in contact with an electrolyte while maintaining structural strength of the rest of the electrode. In certain such examples, the porosity can be configured to maintain a predetermined ESA/GSA ratio during the electrode dissolution. For another example, the porosity can facilitate bonding of the electrode surface to other materials (e.g., coating materials; electrode support structures) due to the combination of enhanced adhesion and mechanical keying between the electrode and the other materials so as to reduce or prevent dislodging of the electrode from the other materials. For still another example, the pores can contain at least one substance (e.g., medicament) and can be configured to controllably introduce (e.g., release; elute) the at least one substance into the recipient's body. In contrast to a completely solid electrode or an electrode with a uniform porosity, certain implementations described herein provide simultaneous optimization of the porosity for multiple functions of the multiple portions of the electrode.

The teachings detailed herein are applicable, in at least some implementations, to any type of implantable medical device (e g, implantable sensory prostheses) configured to apply stimulation signals to a portion of the recipient's body. For example, the implantable medical device can comprise an auditory prosthesis system configured to generate and apply stimulation signals that are perceived by the recipient as sounds (e.g., evoking a hearing percept). Merely for ease of description, apparatus and methods disclosed herein are primarily described with reference to an illustrative auditory prosthesis system, namely a cochlear implant. Examples of other auditory prosthesis systems compatible with certain implementations described herein include but are not limited to: acoustic hearing aids, bone conduction devices (e.g., active and passive transcutaneous bone conduction devices; percutaneous bone conduction devices), middle ear auditory prostheses, direct acoustic stimulators, other electrically simulating auditory prostheses (e.g., auditory brain stimulators), and/or combinations or variations thereof. Examples of other sensory prosthesis systems that are configured to evoke other types of neural or sensory (e.g., sight, tactile, smell, taste) percepts and are compatible with certain implementations described herein include but are not limited to: vestibular devices (e.g., vestibular implants), visual devices (e.g., bionic eyes), visual prostheses (e.g., retinal implants), somatosensory implants, and chemosensory implants.

The teachings detailed herein and/or variations thereof can also be used with a variety of other medical devices that provide a wide range of therapeutic benefits to recipients, patients, or other users. In some implementations, the teachings detailed herein and/or variations thereof can be utilized in other types of implantable medical devices beyond sensory prostheses. For example, apparatus and methods disclosed herein and/or variations thereof can be used with one or more of the following: sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc. Implementations can include any type of medical device that can utilize the teachings detailed herein and/or variations thereof. In addition, the teachings detailed herein and/or variations thereof can also be used with a variety of other non-implantable and/or non-medical devices. For example, apparatus and methods disclosed herein and/or variations thereof can be used with one or more of the following devices comprising at least one electrode in electrical and mechanical communication with another material: chemical sensors; batteries; fuel cells.

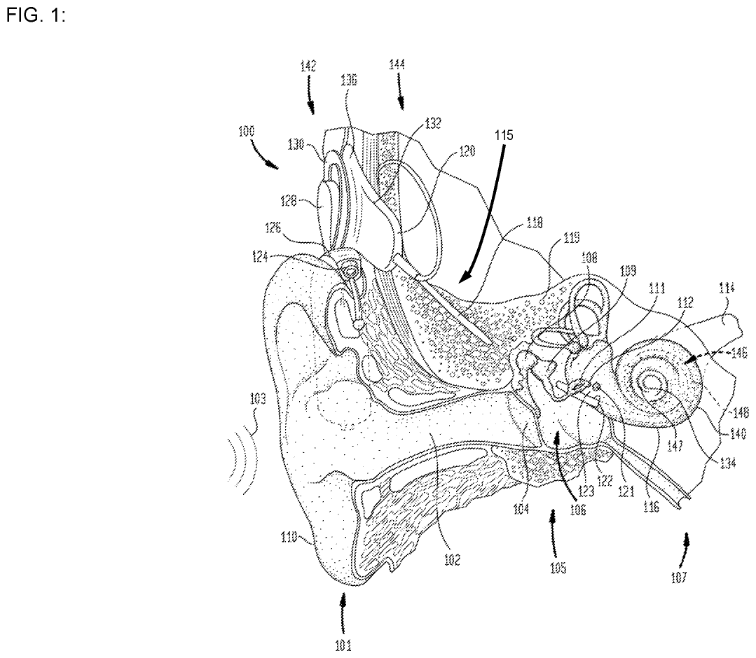

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

The human skull is formed from a number of different bones that support various anatomical features. Illustrated in FIG. 1 is the temporal bone 115 which is situated at the side and base of the recipient's skull (covered by a portion of the recipient's skin/muscle/fat, collectively referred to herein as tissue). For ease of reference, the temporal bone 115 is referred to herein as having a superior portion and a mastoid portion. The superior portion comprises the section of the temporal bone 115 that extends superior to the auricle 110. That is, the superior portion is the section of the temporal bone 115 that forms the side surface of the skull. The mastoid portion, referred to herein simply as the mastoid bone 119, is positioned inferior to the superior portion. The mastoid bone 119 is the section of the temporal bone 115 that surrounds the middle ear 105.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone 115 adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), a mini or micro-BTE unit, an in-the-canal unit that is configured to be located in the recipient's ear canal, a body-worn sound processing unit, etc.

The sound processing unit 126 of certain implementations includes a power source (not shown in FIG. 1)(e.g., battery), a processing module (not shown in FIG. 1)(e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementation of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates stimulation signals (e.g., electrical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation electrodes 148 (e.g., electrical contacts). The stimulation electrodes 148 are longitudinally spaced from one another along a length of the elongate body of the stimulation assembly 118. For example, the stimulation assembly 118 can comprise an array 146 comprising twenty-two (22) stimulation electrodes 148 that are configured to deliver stimulation signals to the cochlea 140. Although the stimulation electrodes 148 of the array 146 can be disposed on the stimulation assembly 118, in most practical applications, the array 146 is integrated into the stimulation assembly 118 (e.g., the stimulation electrodes 148 of the array 146 are disposed in the stimulation assembly 118). As noted, the stimulator unit 120 generates stimulation signals (e.g., electrical signals) which are applied by the stimulation electrodes 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

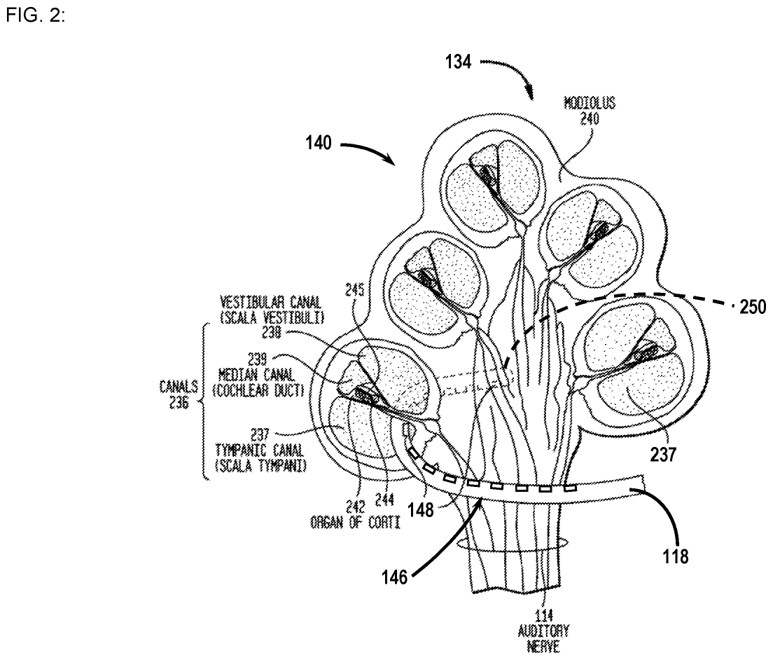

FIG. 2 is cross-sectional view of the cochlea 140 illustrating the stimulating assembly 118 partially implanted therein in accordance with certain implementations described herein. Only a subset of the stimulation electrodes 148 of the stimulation assembly 118 is shown in FIG. 2. The cochlea 140 is a conical spiral structure that comprises three parallel fluid-filled canals or ducts, collectively and generally referred to herein as canals 236. Canals 236 comprise the tympanic canal 237, also referred to as the scala tympani 237, the vestibular canal 238, also referred to as the scala vestibuli 238, and the median canal 239, also referred to as the scala media 239. The cochlea 140 includes the modiolus 240 which is a conical shaped central region around which the cochlea canals 236 spiral. The modiolus 240 consists of spongy bone in which the cochlea nerve cells, sometimes referred to herein as the spiral ganglion cells, are situated. The cochlea canals 236 generally turn 2.5 times around the modiolus 240.

In normal hearing, sound entering the auricle 110 (see, e.g., FIG. 1) causes pressure changes in the cochlea 140 that travel through the fluid-filled tympanic and vestibular canals 237, 238. The organ of Corti 242, which is situated on the basilar membrane 244 in scala media 239, contains rows of hair cells (not shown) which protrude from its surface. Located above the hair cells is the tectoral membrane 245 which moves in response to pressure variations in the fluid-filled tympanic and vestibular canals 237, 238. Small relative movements of the layers of the tectoral membrane 245 are sufficient to cause the hair cells to move, thereby causing the creation of a voltage pulse or action potential which travels along the associated nerve fibers that connect the hair cells with the auditory nerve 114. The auditory nerve 114 relays the impulses to the auditory areas of the brain (not shown) for processing.

Typically, in cochlear implant recipients, some portion of the cochlea 140 (e.g., the hair cells) is damaged such that the cochlea 140 cannot transduce pressure changes into nerve impulses for relay to the brain. As such, the stimulating electrodes 148 of the stimulating assembly 118 are used to directly stimulate the cells to create nerve impulses resulting in perception of a received sound (e.g., to evoke a hearing precept).

To insert the intra-cochlear stimulating assembly 118 into the cochlea 140, an opening (facial recess) is created through the recipient's mastoid bone 119 (see, e.g., FIG. 1) to access the recipient's middle ear cavity 106 (see, e.g., FIG. 1). An opening is then created from the middle ear 106 into the cochlea 140 through, for example, the round window 121, oval window 112, the promontory 123, etc. of the cochlea 140. The stimulating assembly 118 is then gently advanced (e.g., pushed) forward into the cochlea 140 until the stimulating assembly 118 achieves the implanted position. As shown in FIGS. 1 and 2, the stimulating assembly 118 follows the helical shape of the cochlea 140. That is, the stimulating assembly 118 spirals around the modiolus 240.

The effectiveness of the stimulation by the stimulation assembly 118 depends, at least in part, on the place along the basilar membrane 244 where the stimulation is delivered. That is, the cochlea 140 has characteristically been referred to as being “tonotopically mapped,” in that regions of the cochlea 140 toward the basal end are more responsive to high frequency signals, while regions of cochlea 140 toward the apical end are more responsive to low frequency signals. These tonotopical properties of the cochlea 140 are exploited in a cochlear implant by delivering stimulation within a predetermined frequency range to a region of the cochlea 140 that is most sensitive to that particular frequency range. However, this stimulation relies on the particular stimulation electrodes 148 having a final implanted positioned adjacent to a corresponding tonotopic region of the cochlea 140 (e.g., a region of the cochlea 140 that is sensitive to the frequency of sound represented by the stimulation element 148).

To achieve a selected final implanted position, the apical (e.g., distal end/tip) portion 250 of the array 146 is placed at a selected angular position (e.g., angular insertion depth). As used herein, the angular position or angular insertion depth refers to the angular rotation of the apical portion 250 of the array 146 from the cochleostomy 122 (e.g., round window 121) through which the stimulation assembly 118 enters the cochlea 140. In certain implementations, while the stimulation assembly 118 is being implanted (e.g., during a surgical procedure conducted by an operator, such as a medical professional, surgeon, and/or an automated or robotic surgical system), a location and/or an orientation of the array 146 relative to the cochlea 140 (e.g., collectively referred to as the pose of the array 146) is adjusted as the array 146 is advanced and placed into position within the cochlea 140. The goal of the implantation is that the fully-implanted array 146 has an optimal pose in which the array 146 is positioned such that the stimulation electrodes 148 are adjacent to the corresponding tonotopic regions of the cochlea 140. To achieve the optimal pose, the array 146 can follow a trajectory in the cochlea 140 whereby (i) the stimulation electrodes 148 are distributed linearly along an axis of the cochlear duct 239, (ii) the array 146 does not make contact with the basilar membrane 244, and (iii) the stimulation electrodes 148 are in close proximity to the modiolar wall (e.g., if the array 146 is pre-curved) or the stimulation electrodes 148 are distant from the modiolar wall (e.g., if the array 146 is not pre-curved).

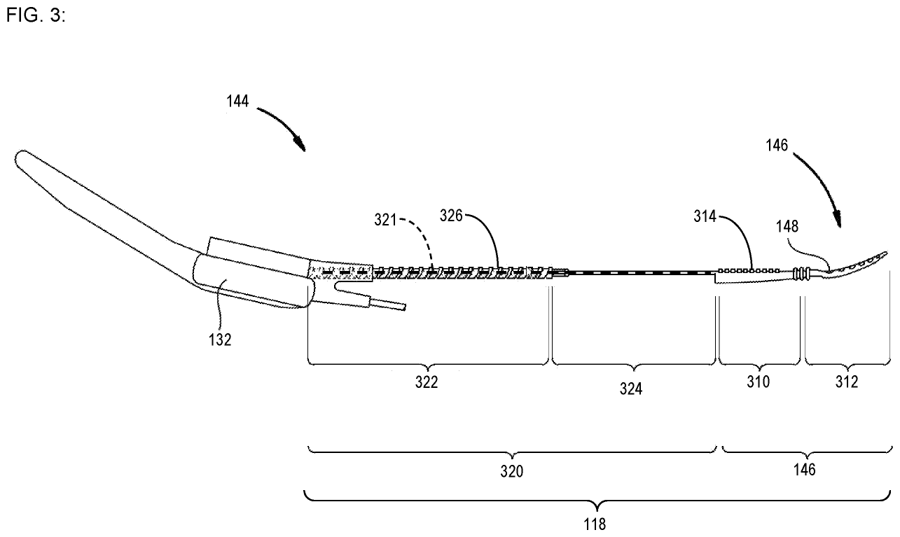

FIG. 3 schematically illustrates a simplified side view of an example internal component 144 comprising at least one stimulation electrode 148 in accordance with certain implementations described herein. The internal component 144 comprises an internal receiver unit 132 which receives encoded signals from an external component 142 of the auditory prosthesis 100 (e.g., cochlear implant system). The internal component 144 terminates in the stimulation assembly 118 that comprises an extra-cochlear region 310 and an intra-cochlear region 312. The intra-cochlear region 312 is configured to be implanted in the recipient's cochlea 140 and has disposed thereon the longitudinally aligned and distally extending array 146 (e.g., electrode array; contact array) comprising a plurality of stimulation electrodes 148. In the example schematically illustrated in FIG. 3, the plurality of stimulation electrodes 148 are configured to apply electrical stimulation to the recipient's body.

In certain implementations, the stimulation assembly 118 comprises a lead region 320 coupling the internal receiver unit 132 to the array 146. In certain implementations, electrical stimulation signals generated by the internal receiver unit 132 are delivered to the array 146 via the lead region 320. The lead region 320 comprises a first portion 322 configured to accommodate movement (e.g., is flexible) and a second portion 324 configured to connect the first portion 322 to the array 146. The first region 322 of certain implementations is configured to prevent the stimulation assembly 118, the lead region 320 and its connection to the internal receiver unit 132, and the array 146 from being damaged due to movement of the internal component 144 (or part of the internal component 144) which may occur, for example, during mastication. In certain implementations, the second region 324 comprises a distinct connection to the first region 322 and/or the array 146, while in certain other implementations, the second region 324 is blended into the first region 322 and/or the array 146. The relative lengths of the stimulation assembly 118, the lead region 320, the first portion 322, the second portion 324, the extra-cochlear region 310, the intra-cochlear region 312, and the array 146 are not shown to scale in FIG. 3.

In certain implementations, the lead region 320 comprises a body 326 and a plurality of signal conduits (e.g., electrical wire leads; not shown) within the body 326. For example, the body 326 can comprise silicone or other biocompatible material in which the signal conduits are embedded (e.g., the body 326 is molded around the signal conduits) or the body 326 can comprise a tube in which the signal conduits are contained (e.g., the tube backfilled with silicone). The signal conduits of certain implementations comprise wires (e.g., platinum; platinum-iridium alloys) having outer diameters that are wavy or helixed around an axis substantially parallel to the longitudinal direction 321 of the lead region 320 (e.g., within the first region 322) and/or are substantially straight and substantially parallel to the longitudinal direction 321 (e.g., within the second region 324). In certain implementations, each of the signal conduits is connected to a corresponding one of the plurality of stimulation electrodes 148 of the array 146.

In certain implementations, the extra-cochlear region 310 is located in the middle ear cavity of the recipient after implantation of the intra-cochlear region 312 into the cochlea 140. Thus, the extra-cochlear region 310 corresponds to a middle ear cavity sub-section of the array 146. In certain implementations, an outer surface of the extra-cochlear region 310 comprises nubs 314 configured to aid in the manipulation of the stimulation assembly 118 during insertion of the intra-cochlear region 312 into the cochlea 140.

Various types of stimulation assemblies 118 are compatible with certain implementations described herein, including short, straight, and peri-modiolar. In certain implementations, the stimulation assembly 118 is a peri-modiolar stimulation assembly 118 having an intra-cochlear region 312 that is configured to adopt a curved configuration during and or after implantation into the recipient's cochlea 140. For example, the intra-cochlear region 312 of the stimulation assembly 118 can be pre-curved to the same general curvature of a cochlea 140. Such peri-modiolar stimulation assemblies 118 are typically held straight by, for example, a stiffening stylet (not shown) or sheath which is removed during implantation, or alternatively by varying material combinations or the use of shape memory materials, so that the stimulation assembly 118 can adopt its curved configuration when in the cochlea 140. Other methods of implantation, as well as other stimulation assemblies 118 which adopt a curved configuration, can also be used.

In certain implementations, the stimulation assembly 118 is a non-perimodiolar (e.g., straight) stimulation assembly 118 or a mid-scala assembly which assumes a mid-scala position during or following implantation. Alternatively, the stimulation assembly 118 of certain implementations comprises a short electrode implanted into at least the basal region. The stimulation assembly 118 can extend towards the apical end of the cochlea 140, referred to as the cochlea apex. In certain implementations, the stimulation assembly 118 is configured to be inserted into the cochlea 140 via a cochleostomy. In certain other implementations, a cochleostomy is formed through the oval window 112, the round window 121, the promontory 123, or through an apical turn of the cochlea 140.

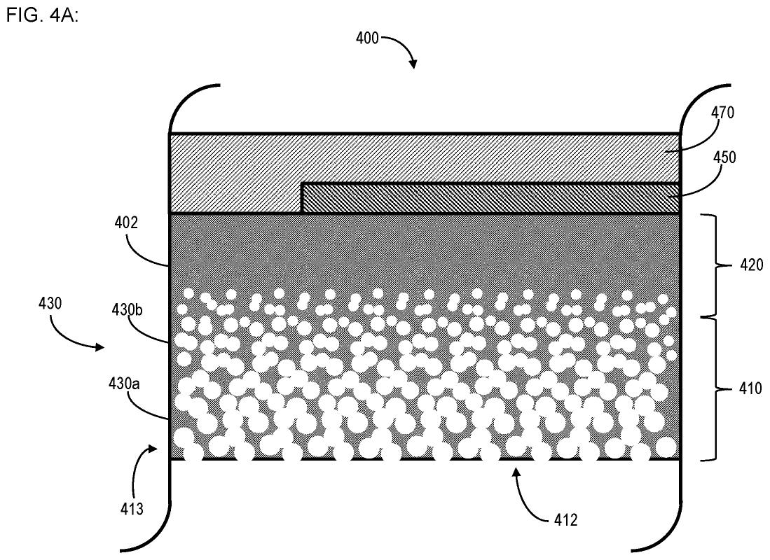

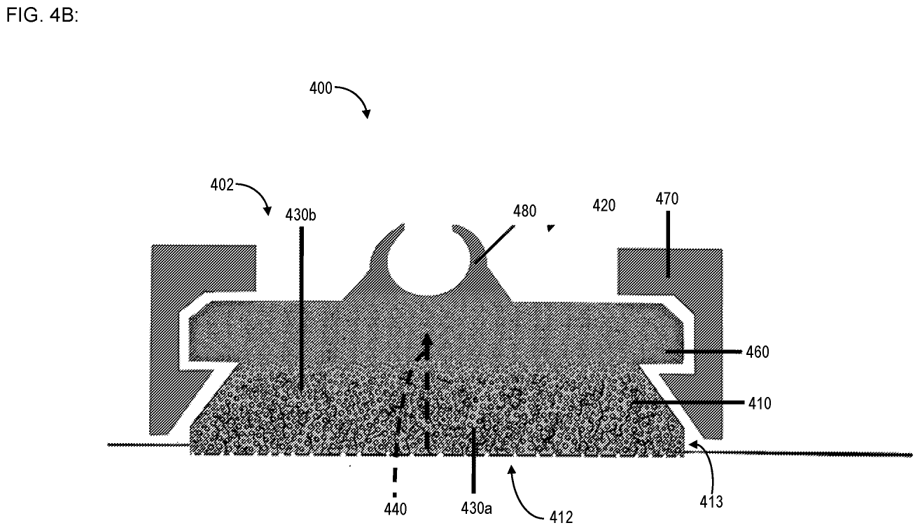

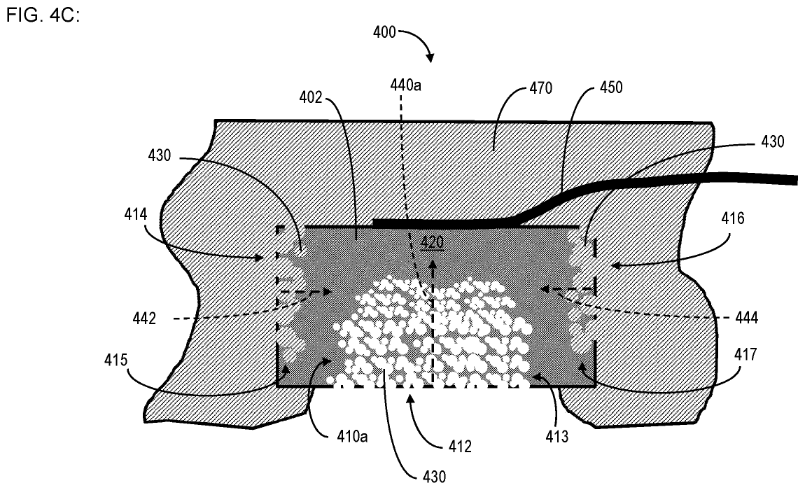

FIGS. 4A-4C schematically illustrate cross-sectional views of various example apparatus 400 in accordance with certain implementations described herein. The apparatus 400 comprises an electrode 402 configured to be implanted on or within a recipient's body. The electrode 402 comprises a first portion 410 having a surface 412 configured to be in electrical communication with the recipient's body. The electrode 402 further comprises a second portion 420 integral with the first portion 410 and in mechanical and electrical communication with the first portion 410. The electrode 402 further comprises a plurality of pores 430 extending from the surface 412 of the first portion 410 to the second portion 420 such that the first portion 410 has a substantially non-uniform porosity along a direction 440 from the surface 412 to the second portion 420.

In certain implementations, the apparatus 400 comprises at least a portion of a medical device (e.g., sensory prosthesis) configured to be implanted on or within a predetermined portion of the recipient's body. The apparatus 400 of certain implementations comprises a single electrode 402 having a substantially non-uniform porosity as described herein, while the apparatus 400 of certain other implementations comprises a plurality of electrodes 402, some or all of which each having a substantially non-uniform porosity as described herein. For example, the plurality of electrodes 402 can comprise a longitudinally aligned and distally extending array 146 of porous stimulation electrodes 148 each having a substantially non-uniform porosity, longitudinally spaced from one another along a length of a stimulation assembly 118, and configured to deliver stimulation signals to the recipient's cochlea 140.

In certain implementations, the electrode 402 is configured to apply electrical stimulation signals to the recipient's body. For example, the electrical stimulation signals can be configured to evoke a neural, sensory, somatosensory, or chemosensory percept (e.g., hearing; sight; tactile; smell; taste; balance; pressure; pain; temperature) and/or to affect (e.g., control) the functioning of a portion of the recipient's body (e.g., cardiac pacemaker or defibrillation signals; autonomic nervous system stimulation signals; brain stimulation signals; muscle stimulation signals; electroporation signals). In certain other implementations, the electrode 402 is configured to receive electrical signals from a portion of the recipient's body. For example, the received electrical signals can be indicative of the functioning of a portion of the recipient's body (e.g., electroencephalogram signals; electrocardiogram signals; electromyograph signals). In certain implementations, the electrode 402 can have one or more dimensions (e.g., width; length; height; thickness) in a range of 200 microns to 1 millimeter (e.g., in a range of 300 microns to 400 microns).

In certain implementations, the first portion 410 and the second portion 420 are parts of a single (e.g., unitary; integrated; monolithic) element. In certain implementations, each of the first portion 410 and the second portion 420 of the electrode 402 comprises at least one electrically conductive material selected from the group consisting of: metal; noble metal; non-noble metal; platinum; palladium; ruthenium; rhodium; osmium; iridium; titanium; gold; alloys comprising one or more of the foregoing; composites comprising one or more of the foregoing. For example, the electrically conductive material can comprise platinum-iridium alloy with an iridium content in a range of l0 wt % to 30 wt %. In certain implementations, the first portion 410 comprises at least one first electrically conductive material (e.g., at least one first metal) and the second portion 420 comprises at least one second electrically conductive material (e.g., at least one second metal) that is the same as the at least one first electrically conductive material or that is different than the at least one first electrically conductive material. In certain implementations, the composition of the at least one first electrically conductive material can vary through the first portion 410 and/or the composition of the at least one second electrically conductive material can vary through the second portion 420.

The first portion 410 of certain implementations comprises a first region (e.g., surface region 413) at least partially bounded by the surface 412 and comprising at least some of the plurality of pores 430. The second portion 420 of certain implementations comprises a second region (e.g., non-surface region) spaced away from the surface 412. For example, the second portion 420 can be spaced from some or all of the surfaces of the electrode 402. For another example, the second portion 420 can be at least partially bounded by at least one surface (e.g., a side or back surface of the electrode 402) that is different from the surface 412 (e.g., a front surface of the electrode 402) and/or that is not in direct mechanical contact with the recipient's body. In certain implementations, the second portion 420 comprises at least some of the plurality of pores 430, while in certain other implementations, the second portion 420 is substantially non-porous (e.g., having a porosity substantially equal to zero). In certain implementations, the first portion 410 has a first thickness extending from the surface 412 such that the second portion 420 has a second thickness sufficient to provide adequate mechanical strength for operation of the electrode 402. For example, for an electrode 402 having a total thickness of 100 microns, the first thickness of the first portion 410 can be in a range of 60 microns to 70 microns and the second thickness of the second portion 420 can be in a range of 30 microns to 40 microns.

In certain implementations, the surface 412 is configured to be in electrical communication and in direct mechanical contact with tissue, cells, fluid, and/or another portion of the recipient's body. In certain implementations, the surface 412 is substantially flat (e.g., planar), while in certain other implementations, the surface 412 is curved or irregular (e.g., non-planar). The In certain implementations, the surface 412 of the first portion 410 of the electrode 402 extends over an area in a range of 0.01 mm² to 1 mm² (e.g., in a range of 0.025 mm² to 0.5 mm²).

In certain implementations, the apparatus 400 further comprises at least one electrical conduit 450 (e.g., one or more wires or leads) in mechanical and electrical communication with the second portion 420 of the electrode 402. The electrical conduit 450 can be configured to transmit electrical signals between the electrode 402 and a controller (e.g., processor; digital signal processor; microcontroller core; application-specific integrated circuit; circuitry) of the apparatus 400. For example, the controller can be configured to generate electrical signals to be delivered by the electrode 402 to the recipient's body and/or to receive electrical signals received by the electrode 402 from the recipient's body. As schematically illustrated by FIGS. 4A and 4C, the electrical conduit 450 of certain implementations is be affixed (e.g., bonded; welded; crimped) to the second portion 420 of the electrode 402. The electrical conduit 450 and the electrode 402 of certain other implementations are parts of a single (e.g., unitary; integrated) element. In certain implementations, the electrical conduit 450 comprises at least one material selected from the group consisting of: metal; noble metal; non-noble metal; platinum; palladium; ruthenium; rhodium; osmium; iridium; titanium; gold; alloys of one or more of the foregoing; composites of one or more of the foregoing. The electrical conduit 450 can have a width (e.g., outer diameter) that is in a range of 0.05 millimeter to 0.3 millimeter (e.g., in a range of 0.1 millimeter to 0.2 millimeter).

In certain implementations, as schematically illustrated in FIGS. 4A-4C, the apparatus 400 further comprises an electrically insulative (e.g., non-electrically conductive) element 470 configured to support the electrode 402 and/or to electrically isolate the electrode 402 and/or the electrical conduit 450 from portions of the surrounding environment. For example, the electrically insulative element 470 can comprise at least one biocompatible material (e.g., polymer; polyether ether ketone (PEEK); elastomer; silicone; rubber; ceramic) and/or can be a portion of a body in which the electrode 402 and/or the electrical conduit 450 are embedded or contained (e.g., hermetically sealed within). In certain implementations, the electrically insulative element 470 comprises one or more recesses and/or protrusions configured to mate with one or more protrusions 460 and/or recesses of the electrode 402 (see, e.g., FIG. 4B).

In certain implementations, the pores 430 have varying sizes, varying shapes (e.g., substantially spherical, elongated, regular, irregular, symmetric, asymmetric, geometric, non-geometric), and/or varying numbers (e.g., more pores or fewer pores per unit area or unit volume). At least some of the pores 430 of certain implementations are open and/or interconnected with one another to provide fluid communication across at least some of the first portion 410 to the surface 412. At least some of the pores 430 of certain implementations are closed (e.g., discrete volumes that are not interconnected and do not provide fluid communication therebetween). In certain implementations, for an electrode 402 fabricated using laser sintering of metal powder particles, the pore sizes and pore density can be dependent on the metal powder particle size. For example, for an electrode 402 having a diameter less than 500 microns, the metal powder particle size can be larger than 1 micron (e.g., in a range of 3 microns to 5 microns), and the minimum pore size can be of the same order or smaller. For another example, for suitability for integration of the surface 412 with a silicone carrier or polymer coating, the pore sizes can be in a range of 10 microns to 20 microns.

In certain implementations, the porosity of a region of the electrode 402 is defined as a percentage or fraction of a volume of the pores within the region to a total volume of the region. The porosity of a region is dependent upon the sizes of the pores in the region (e.g., larger pores corresponding to higher porosities and smaller pores corresponding to lower porosities) and upon the number of pores in the region (e.g., more pores corresponding to higher porosities and fewer pores corresponding to lower porosities). The porosity of a region can be expressed as an average porosity over a total volume of the region. For example, a first porosity of the first portion 410 can be expressed as an average porosity over the whole volume of the first portion 410 and a second porosity of the second portion 420 can be expressed as an average porosity over the whole volume of the second portion 420. For example, the first porosity of the first portion 410 can be in a range of 40% to 60% (e.g., about 50% for suitability for integration of the surface 412 with a silicone carrier or polymer coating) and the second porosity of the second portion 420 can be in a range of 0% to 10% (e.g., for suitability for structural stability). The close packing of identical spheres can result in densities in a range of 64% to 74%, which corresponds to a porosity in a range of 26% to 34%, and the distribution of particle sizes in metal powders can result in higher densities and lower porosities (e.g., less than 26%). These porosity values are smaller than porosity values that may be expected for facilitating tissue ingrowth with the electrode 402 (e.g., fabricated using metal particles with diameters of the order of 200 microns)(see, e.g., M. S. Hirshorn et al., “Effect of Pore Size on Threshold and Impedance of Pacemaker Electrodes,” in “Cardiac Pacing,” Steinbach K. (eds.), Steinkopff, Heidelberg; https://doi.org/10.1007/978-3-642-72367-4_57 (1983)). The porosity of a region can also be expressed as a function of position within the region (e.g., as an average porosity over sub-areas or sub-volumes within the region). A porosity that is substantially unchanging as a function of position within the region (e.g., by less than 10%; by less than 5%; by less than 2%) can be referred to as a substantially uniform porosity, and a porosity that substantially varies as a function of position within the region (e.g., by more than 50%; by more than 40%; by more than 30%; by more than 20%; by more than 10%) can be referred to as a substantially non-uniform porosity. For example, for an electrode 402 having a thickness of 100 microns or less and fabricated using metal powder particle sizes in a range of 3 microns to 5 microns, the porosity can have a gradient such that the porosity varies from a high value (e.g., greater than 50%) to a low value (e.g., approaching zero) over a distance of about 10 microns. The mass density of a region of the electrode 402 is inversely proportional to the porosity of the region since the pores have a lower mass density than does the solid materials of the electrode 402.

As schematically illustrated by FIG. 4A, in certain implementations, a porosity of the electrode 402 in the surface region 413 of the electrode 402 is greater than in a non-surface region of the electrode 402 (e.g., the first portion 410 has a first average porosity and the second portion 420 has a second average porosity less than the first average porosity). In certain implementations, the porosity of the first portion 410 can be substantially non-uniform with a first value at the surface 412 and a second value spaced from the surface 412 (e.g., adjacent to the second portion 420), the second value less than the first value. For example, the average porosity of the first portion 410 as a function of position along the direction 440 (e.g., throughout the volume of the first portion 410) can be non-zero, graded (e.g., having a non-zero slope or gradient) and substantially continuous (e.g., having a substantially non-infinite slope or gradient) from the surface 412 of the first portion 410 to the second portion 420. As schematically illustrated by FIG. 4A, the pores 430a at a first position that is a first distance from the surface 412 can have larger sizes and/or be more numerous than the pores 430b at a second position that is a second distance from the surface 412, the second distance larger than the first distance, and the sizes and/or numbers of pores 430 between the first and second positions can vary substantially continuously between the first position and the second position. In certain implementations, the substantially non-uniform porosity is monotonically decreasing along the direction 440 (e.g., substantially perpendicular to the surface 412) from the surface 412 to the second portion 420. For example, the porosity averaged over planar areas substantially parallel to the surface 412 can be monotonically decreasing along the direction 440 from a maximum value at the surface 412 to the second portion 420 (e.g., approaching a porosity of zero).

In certain implementations, the substantially non-uniform porosity of the plurality of pores 430 is configured to facilitate structural integrity of the electrode 402 and/or the mechanical strength of the support of the electrode 402 by the surrounding portions of the apparatus 400. For example, the first portion 410 can comprise a surface region 413 bounded at least in part by the surface 412 and having a higher porosity and lower mass density, and the porosity of the first portion 410 can be graded along the direction 440 (e.g., substantially perpendicular to the surface 412) towards the second portion 420 having a lower porosity and higher mass density compared to that of the first portion 410. In certain implementations, the lower porosity and higher mass density of the second portion 420 reduces (e.g., prevents) material fracture and/or crumbling of the electrode 402 as compared to a second portion 420 having a porosity and mass density substantially equal to that of the first portion 410. In certain implementations, the substantially non-uniform porosity is substantially continuously graded throughout the first portion 410 to the second portion 420 (e.g., has a substantially continuous gradation from the surface region to the non-surface region) such that the substantially non-uniform porosity does not have a sharp change in morphology (e.g., not having s substantially infinite slope or gradient), thereby reducing (e.g. preventing) the porous surface region 413 from delaminating from the electrode 402.

As schematically illustrated by FIG. 4A, a region of the second portion 420 can be configured to have sufficient structural strength to be affixed to at least one electrical conduit 450 (e.g., one or more wires or leads) such that the at least one electrical conduit 450 is in electrical and mechanical communication with the electrode 402 (e.g., and to remain being so affixed throughout the operational lifetime of the apparatus 400). As schematically illustrated by FIG. 4B, the second portion 420 of the electrode 402 of certain implementations can further comprise other structural elements configured to be in mechanical communication with other components of the apparatus 400. For example, the second portion 420 of FIG. 4B comprises one or more protrusions 460 (e.g., overhangs) and/or recesses (e.g., grooves) configured to mate with one or more recesses and/or protrusions of an electrically insulative (e.g., non-electrically conductive) element 470 of the apparatus 400 (e.g., electrode array substrate), the electrically insulative element 470 configured to support the electrode 402 and/or to electrically isolate the electrode 402 from portions of the surrounding environment. For another example, the second portion 420 of FIG. 4B comprises a plastically deformable portion 480 configured to be crimped onto or at least partially around a corresponding electrical conduit 450 such that the electrode 402 is in electrical communication with the electrical conduit 450. The substantially non-uniform porosity of certain implementations is configured to prevent fluid from the environment that is in fluid communication with the surface 412 from reaching the second portion 420 of the electrode 402, thereby preventing erosion of the electrode material from the second portion 420 which would otherwise compromise the structural integrity and/or strength of the electrode 402 and/or the bonding of the electrode 402 to the at least one electrical conduit 450.

In certain implementations, the substantially non-uniform porosity of the plurality of pores 430 is configured to facilitate mechanical bonding of the electrode 402 with other materials (e.g., coatings). In certain implementations, the surface 412 and the surface region 413 have a substantially non-uniform porosity sufficient to facilitate bonding between the electrode 402 and a coating configured to enhance the charge injection capacity of the electrode 402 and/or to reduce the susceptibility of the electrode 402 to dissolution. For example, an electrically conductive polymer-based coating (e.g., poly(3, 4-ethylenedioxythiophene (PEDOT)) can be applied to the surface 412 having the substantially non-uniform porosity during fabrication of the electrode 402. In certain implementations, the use of a coating can allow the thickness of the electrode 402 to be reduced (e.g., metal having a thickness in a range of 25 microns to 50 microns with polymer-based coating) compared to an uncoated electrode 402 that relies on the porous surface region 413 for electrochemical surface area. In certain implementations, the electrode 402 has an electrically conductive polymer-based coating on a first portion 410 having a first thickness of about 10 microns and a second portion 420 having a second thickness in a range of 15 microns to 40 microns. The bonding between the electrode 402 and the coating can be strengthened via improved adhesion between the coating and the surface 412 and/or mechanical keying of the coating material within the pores 430 of the surface 412 and the surface region 413 such that delamination of the coating from the electrode 402 is lessened throughout the operational lifetime of the apparatus 400 as compared to such a coating on a surface 412 and surface region 413 without the pores 430. The substantially non-uniform porosity of certain implementations described herein can be used to improve the bonding between the electrode 402 and other non-polymer-based electrode coatings (e.g., platinum black; platinum grey; iridium oxide; titanium nitride) that are known to otherwise exhibit reduced mechanical stability, poor adhesion to the underlying surface, and high risk of delamination from the electrode 402 over the operational lifetime of the apparatus 400.

In certain implementations, at least one surface and at least one corresponding surface region can have a substantially non-uniform porosity sufficient to facilitate bonding between the electrode 402 and a portion of the apparatus 400 configured to support the electrode 402 and/or to at least partially protect (e.g., electrically isolate; chemically isolate) the electrode 402 from the surrounding environment. For example, as schematically illustrated in FIG. 4C, the electrode 402 comprises a first side surface 414 and a corresponding surface region 415 with a substantially non-uniform porosity along a direction 442 from the first side surface 414 to a non-surface region (e.g., second portion 420) of the electrode 402. The electrode 402 of FIG. 4C further comprises a second side surface 416 and a corresponding surface region 417 with a substantially non-uniform porosity along a direction 444 from the second side surface 416 to a non-surface region (e.g., second portion 420) of the electrode 402. The apparatus 400 further comprises an electrically insulative element 470 comprising at least one material (e.g., polymer; polyether ether ketone (PEEK); elastomer; silicone; rubber) that can be applied in a fluid state to the electrode 402 and can then harden into a solid state to at least partially protect the electrode 402 from the surrounding environment and/or to support the electrode 402 within the apparatus 400. While in the liquid state, the material can be applied (e.g., injected) onto and/or into the electrode 402 to flow through the surfaces 414, 416 via the pores 430 into the corresponding surface regions 415, 417. The material can then be allowed to harden (e.g., cure) and bond with the electrode 402 and form the electrically insulative element 470. The bonding can be strengthened via improved adhesion between the material and the surfaces 414, 416 and/or mechanical keying of the material within the pores 430 of the surfaces 414, 416 and the corresponding surface regions 415, 417.

In certain implementations, the plurality of pores 430 are configured to facilitate delivery of electrical signals to the environment in fluid communication with the surface 412 of the electrode 402 throughout the operational lifetime of the apparatus 400 during which the electrode 402 is implanted on or within the recipient's body. In certain implementations, the substantially non-uniform porosity of the plurality of pores 430 (e.g., adjacent to and including the surface 412) is configured to provide a sufficiently large ratio of the electrochemical surface area (ESA) of the surface 412 to the geometric surface area (GSA) of the surface 412 (e.g., the ESA/GSA ratio is greater than one) such that the electrode 402 is configured to inject a higher electric charge without inducing water hydrolysis and/or electrode dissolution as compared to an electrode with the same materials and overall dimensions but with a lower ESA/GSA ratio. For example, ESA/GSA ratios for an electrode 402 fabricated by laser sintering of metal powder particles can be in a range of 3 to 8, which is comparable to ESA/GSA ratios resulting from surface roughening (e.g., via mechanical means or via laser) which can allow 3 to 8 times the charge to be delivered without increase in dissolution rate compared to a smooth electrode of the same geometric surface area. In certain implementations, the substantially non-uniform porosity of the plurality of pores 430 (e.g., adjacent to and including the surface 412) is configured to provide a sufficiently large ESA/GSA ratio such that the electrode 402 is configured to have lower impedance and/or lower polarization during pulsing as compared to an electrode with the same materials and overall dimensions but with a lower ESA/GSA ratio. Polarization can be inversely proportional to surface area and can account for 20%-50% of the total impedance. The substantially non-uniform porosity can be used to increase the ESA/GSA ratio with or without other means for increasing the ESA/GSA ratio (e.g., roughening the surface 412; coating the electrode 402 with porous materials such as platinum black, sputtered iridium oxide, and fractal titanium nitride.

In certain implementations, the substantially non-uniform porosity is configured to remain substantially constant (e.g., substantially unchanged; changed by less than 20%, less than 10%, or less than 5%) during dissolution of the electrode 402 over the operational lifetime of the apparatus 400. In certain implementations in which the plurality of pores 430 comprises cells that are open and/or interconnected (e.g., pores 430 that are in fluid communication with the surrounding environment and/or with one another), the substantially non-uniform porosity can be substantially continuous and monotonically decreasing as a function of distance from the surface 412 (e.g., having a substantially continuous gradation from the surface region to the non-surface region) such that the ESA/GSA ratio remains substantially constant (e.g., substantially unchanged; changed by less than 20%, less than 10%, or less than 5%) while the electrode 402 undergoes dissolution (e.g., dissolves away) over the operational lifetime of the apparatus 400 (e.g., while the electrode 402 is implanted on or within the recipient). For example, as the electrode material at the surface 412 dissolves away, thereby exposing new electrode material at the surface 412, electrode material at least partially bounding pores 430 deeper within the electrode 402 also dissolves away, such that the substantially non-uniform porosity of the first portion 410 and the ESA/GSA ratio of the surface 412 remains substantially constant. Once the porous first portion 410 of the electrode 402 is completely dissolved and the second portion 420 becomes exposed to tissue, the ESA/GSA ratio can be substantially lower, charge density can be substantially higher, and there can be a runaway effect of increasing dissolution rate for the remainder of the life of the electrode 402. In certain other implementations, the plurality of pores 430 comprises cells that are closed (e.g., pores 430 that are not in fluid communication with the surrounding environment or with one another) but which become open or interconnected cells as the electrode material dissolves and the environment becomes fluidly connected with the interiors of the previously-closed cells. In certain such implementations, the substantially non-uniform porosity can be substantially continuous and configured such that the ESA/GSA ratio remains substantially constant (e.g., substantially unchanged; changed by less than 20%, less than 10%, or less than 5%) while the electrode 402 undergoes dissolution (e.g., dissolves away) over the operational lifetime of the apparatus 400 (e.g., while the electrode 402 is implanted on or within the recipient).

In certain implementations, the plurality of pores 430 is configured to deliver at least one substance to the environment in fluid communication with the surface 412 of the electrode 402. In certain such implementations, the plurality of pores 430 has a substantially non-uniform porosity (e.g., a non-zero gradient in pore size), while in certain other such implementations, the plurality of pores 430 has a substantially uniform porosity (e.g., a zero gradient in pore size).

In certain implementations, the plurality of pores 430 (e.g., open cells and/or interconnected cells adjacent to and including the surface 412) are configured to contain at least one medicament (e.g., a drug; a drug-containing substance; a liquid). Examples of medicaments compatible with certain implementations described herein include but are not limited to: a neurotrophin (e.g., neurotrophin-3 (NT-3); brain-derived neurotrophic factor (BDNF)); a steroid (e.g., dexamethasone); other therapeutic agent. The neurotrphin can be used to promote neuron growth towards the electrode 402 and to establish a strong neuroprosthetic interface with dendritic processes integrated into the surface 412 for improved sensitivity and spatial selectivity of the stimulation generated by the electrode 402.

In certain implementations, the plurality of pores 430 is configured such that, after the surface 412 of the electrode 402 is placed in fluid communication with the environment (e.g., after the electrode 402 is implanted within or on the recipient's body), the at least one substance can be controllably released over time from the pores 430 to the environment. For example, the electrode 402 can comprise at least one first material covering the pores 430 at the surface 412 such that the at least one substance within the pores 430 is blocked from entering the environment. The at least one first material can be configured to respond to an electrical voltage and/or current applied to the at least one first material by dissolving, thereby allowing the at least one substance to leave the pores 430 and enter the environment through the surface 412. For another example, the at least one substance within the pores 430 can comprise at least one second material configured to have at least one property (e.g., viscosity; surface tension; diffusion coefficient) responsive to electrical voltage and/or current applied to the at least one second material such that, in absence of the electrical voltage and/or current, the at least one substance remains within the pores 430, and in the presence of the electrical voltage and/or current, the at least one substance leaves the pores 430 and enters the environment through the surface 412. In certain such implementations, the apparatus 400 is configured to deliver the at least one substance to the environment upon demand by applying the electrical voltage and/or current to the at least one first material or the at least one second material in response to a detected event indicative of demand of the at least one substance (e.g., a rise in an impedance of the electrode 402 indicative of an inflammatory event and used to trigger delivery of an anti-inflammatory medicament).

In certain implementations, a porosity of open cells of the plurality of pores 430 is tailored to allow a liquid from the environment (e.g., perilymph) to reach an entrance of a reservoir containing the at least one substance within or behind the electrode 402, the entrance comprising a polymer material (e.g., hydrogel) responsive to an electrical voltage and/or current applied to the polymer material by dissolving, thereby allowing the at least one substance to enter the liquid. In certain other implementations, closed cells of the plurality of pores 430 are loaded with the at least one substance to be released as the closed cells are opened and/or exposed to the environment due to dissolution of the electrode 402 (e.g., for a therapeutic benefit; to inhibit dissolution).

In certain implementations, the electrode 402 can have different substantially non-uniform porosities in different regions of the electrode 402 to provide simultaneous optimization of the porosity for the different functions of the different regions. For example, as schematically illustrated by FIG. 4C, the substantially non-uniform porosity in proximity to the surface 412 in fluid communication with the environment can be tailored for improved (e.g., optimized) electrical performance of the electrode 402 and/or controlled substance introduction to the environment from the electrode 402 while the substantially non-uniform porosity in proximity to the surface 414 and/or surface 416 is simultaneously tailored for improved (e.g., optimized) bonding to other materials.

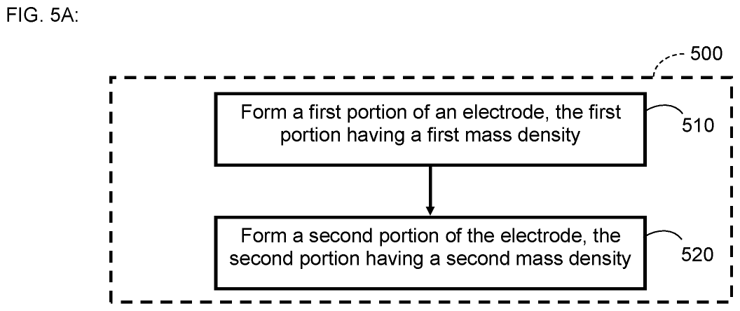

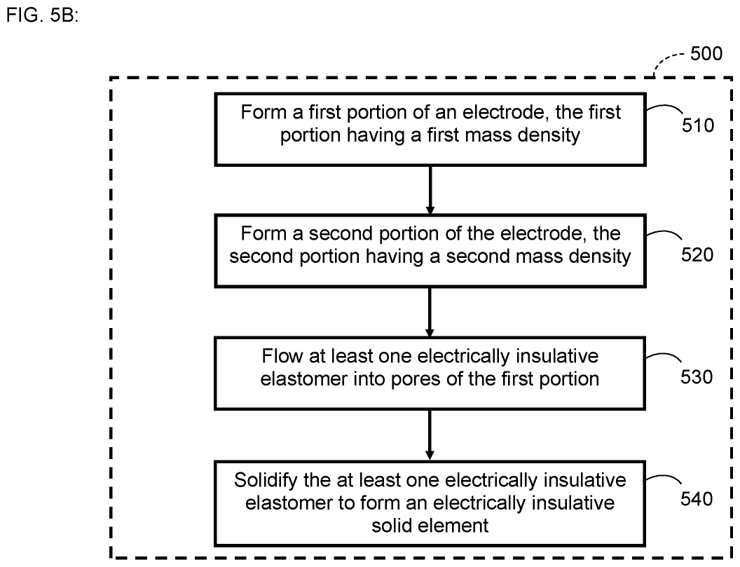

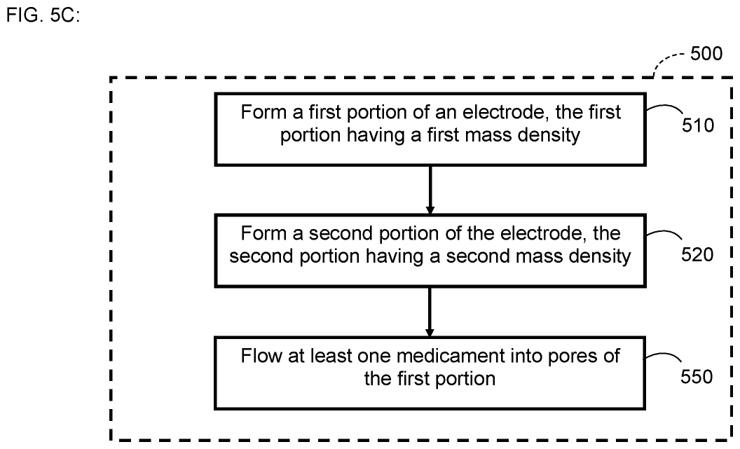

FIGS. 5A-5C are flow diagrams of an example method 500 in accordance with certain implementations described herein. In an operational block 510, the method 500 comprises forming a first portion 410 of an electrode 402, the first portion 410 comprising an electrically conductive material and having a first mass density. In an operational block 520, the method 500 further comprises forming a second portion 420 of the electrode 402, the second portion 420 comprising the electrically conductive material, being integral with the first portion 410, and having a second mass density greater than the first mass density. In certain implementations, forming the first portion 410 and/or forming the second portion 420 are performed using additive manufacturing (e.g., three-dimensional metal printing; laser micro-sintering) to produce the first mass density and/or the second mass density.

In certain implementations, as shown in FIG. 5B, in an operational block 530, the method 500 further comprises flowing at least one electrically insulative (e.g., non-electrically conductive) elastomer into pores 430 of the first portion 410 and overlaying the first portion 410. In an operational block 540, the method 500 further comprises solidifying the at least one non-electrically conductive elastomer to form an electrically insulative solid element 470 mechanically bonded with the first portion 410. In certain implementations, as shown in FIG. 5C, the electrode 402 is configured to be implanted on or within a recipient's body, and in an operational block 550, the method 500 further comprises flowing at least one medicament into pores 430 of the first portion 410. The at least one medicament is configured to controllably flow out of the pores 430 into the recipient's body while the electrode 402 is implanted on or within the recipient's body.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from having at least a portion of the received power available for use by the implanted device during time periods in which the at least one power storage device of the implanted device unable to provide electrical power for operation of the implantable medical device.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
an electrode configured to be implanted on or within a recipient's body, the electrode comprising:
a first portion having a surface configured to be in electrical communication with the recipient's body;
a second portion integral with the first portion and in mechanical and electrical communication with the first portion; and
a plurality of pores extending from the surface of the first portion to the second portion such that the first portion has a substantially non-uniform porosity along a direction from the surface to the second portion.

2. The apparatus of claim 1, wherein the substantially non-uniform porosity is monotonically decreasing along the direction from the surface to the second portion.

3. The apparatus of claim 1, wherein the substantially non-uniform porosity has a first value at the surface and a second value at the second portion, the second value less than the first value.

4. The apparatus of claim 1, wherein the substantially non-uniform porosity has a gradient as a function of position along the direction, the gradient being non-zero and substantially continuous along the direction from the surface to the second portion.

5. The apparatus of claim 4, wherein the direction is substantially perpendicular to the surface and extends towards the second portion.

6. The apparatus of claim 1, wherein the first portion and the second portion comprise at least one metal.

7. The apparatus of claim 1, wherein at least some of the pores are open and/or interconnected with one another to provide fluid communication across at least some of the first portion to the surface.

8. The apparatus of claim 1, wherein the apparatus further comprises an electrical conduit in mechanical and electrical communication with the second portion, the electrical conduit configured to transmit electrical signals between the electrode and a controller of the apparatus.

9. The apparatus of claim 1, wherein the apparatus further comprises a non-electrically conductive material configured to support the electrode.

10. The apparatus of claim 9, wherein the material comprises silicone.

11. The apparatus of claim 9, wherein the material comprises one or more protrusions and/or recesses configured to mate with one or more recesses and/or protrusions of the electrode.

12. A method comprising:
forming a first portion of an electrode, the first portion comprising an electrically conductive material and having a first mass density; and
forming a second portion of the electrode, the second portion comprising the electrically conductive material, being integral with the first portion, and having a second mass density greater than the first mass density.

13. The method of claim 12, wherein forming the first portion and forming the second portion are performed using additive manufacturing to produce the first mass density and the second mass density.

14. The method of claim 12, wherein the electrically conductive material comprises at least one metal, metal alloy, or metal composite.

15. The method of claim 12, further comprising flowing at least one non-electrically conductive elastomer into pores of the first portion and overlaying the first portion and solidifying the at least one electrically insulative elastomer to form an electrically insulative solid element mechanically bonded with the first portion.

16. The method of claim 12, wherein the electrode is configured to be implanted on or within a recipient's body, the method further comprising flowing at least one medicament into pores of the first portion, the at least one medicament configured to controllably flow out of the pores while the electrode is implanted on or within the recipient's body.

17. An apparatus comprising:
a porous electrode configured to be implanted on or within a recipient, the porous electrode having a surface region with a ratio of electrochemical surface area (ESA) to geometric surface area (GSA) greater than one, the surface region configured to undergo dissolution over time while the porous electrode is implanted on or within the recipient with the ratio being substantially unchanged by the dissolution.

18. The apparatus of claim 17, wherein a porosity of the porous electrode in the surface region is greater than in a non-surface region of the porous electrode.

19. The apparatus of claim 18, wherein the pores are open and/or interconnected and the porosity has a substantially continuous gradation as a function of distance from the surface region to the non-surface region.

20. The apparatus of claim 18, wherein the pores are closed and the porosity is substantially continuous.

21. An electrode comprising:
a porous surface configured to be in electrical communication with a surrounding environment;
a porous first metal portion at least partially bounded by the porous surface; and
a substantially non-porous second metal portion integral with the porous first metal portion and in mechanical and electrical communication with the porous first metal portion,
the porous first metal portion having a porosity along a distance in a direction substantially perpendicular to the surface and extending from the porous surface to the substantially non-porous second portion, the porosity varying by more than 10% as a function of position along the distance, monotonically decreasing along the distance, and having a non-zero and substantially continuous gradient as a function of position along the distance.

22. The electrode of claim 21, wherein the second portion is in mechanical and electrical communication with at least one electrical conduit configured to transmit electrical signals to and/or from the electrode.

23. The electrode of claim 21, wherein the porous surface has a ratio of electrochemical surface area (ESA) to geometric surface area (GSA) greater than one.

24. The electrode of claim 21, wherein the porous first metal portion comprises at least one medicament within pores of the porous first metal portion.

25. The electrode of claim 21, wherein the porous first metal portion comprises within pores of the porous first metal portion, the at least one non-electrically conductive elastomer mechanically bonded with the porous first metal portion.
